(19) United States
(12) Patent Application Publication
(10) Pub. No.: US 2016/0207679 A1
(43) Pub. Date: Jul. 21, 2016

(54) LABEL SYSTEM FOR INGESTIBLE PRODUCTS

(71) Applicant: Damon JACKSON, New York, NY (US)

(72) Inventor: Damon JACKSON, New York, NY (US)

(21) Appl. No.: 15/001,573

(22) Filed: Jan. 20, 2016

Related U.S. Application Data
(60) Provisional application No. 62/106,058, filed on Jan. 21, 2015.

(51) Int. Cl. B65D 65/46; A61K 9/00; B65D 77/28; A61K 36/185; G09F 3/02; B65D 85/00

(52) U.S. Cl. CPC B65D 65/463; G09F 3/02; B65D 85/70; B65D 77/28; A61K 36/185; A61K 9/0056; G09F 2003/0283

(57) ABSTRACT

A labeling system embodying a label format for ingestible products and method for preventing accidental and/or over-ingestion of cannabis infused products is provided. The system facilitates identifying the THC and CBD levels of cannabis infused products, providing a clear edible warning label that can be placed directly on the infused edible itself; providing visible identification of cannabis infused food products; and providing descriptive content of cannabis-related information.

### CROSS-REFERENCE TO RELATED APPLICATION

This application claims the benefit of priority of U.S. provisional application No. 62/106,058, filed 21 Jan. 2015, the contents of which are herein incorporated by reference.

### BACKGROUND OF THE INVENTION

The present invention relates to consumer safety and, more particularly, to a label system for ingestible products, wherein the label system embodies a label format and method for preventing accidental and/or over-ingestion of cannabis infused products.

There is a major risk of mistaking cannabis-infused products as regular food products after the infused product's packaging is removed. Particularly because cannabis-infused products are typically designed and shaped to look like other non-cannabis food products that do not present a concern of accidental or over-ingestion. There is currently no “direct-on” label product designed for the ‘infused edibles’ industry that meets regulatory compliance requirements, and as a result leaving increased potential for accidental and/or over-ingestion of infused food products.

As can be seen, there is a need for a labeling system embodying a label format for ingestible products and method for preventing accidental and/or over-ingestion of cannabis infused products, thereby identifying the THC and CBD levels of cannabis infused products, providing a clear edible warning label that can be placed directly on the infused edible itself; providing visible identification of cannabis infused food products; and providing descriptive content of cannabis-related information.

This invention is primarily directed to those products infused with cannabis that are ingestible. The present invention, a direct-on label format system, offers an edible label providing informational space directly on cannabis infused product that will allow consumers and other parties to visually verify that the specifications of the cannabis-related information infused in the product. These edible labels represent information and may include numbers, letters, color and other indicia, etc. This system provides the fastest, easiest system to reduce unacceptable errors and failure to warn, as well as removing inhibitions of implementation of cannabis-infused product error-prevention procedures.

The label format of the present invention may provide legibility of the cannabis-related specifics; visual verification for THC and CBD potency therein; visual representation of relevant regulatory compliance; predetermined code numbers for obtaining all of the specific properties of the cannabis; UPC bar code(s) for confirming cannabis for verification procedures as well as constant inventory management; and is a solution for error-prevention procedures in cannabis dispensing.

### SUMMARY OF THE INVENTION

In one aspect of the present invention, a labeling system for preventing accidental and/or undesirable ingestion of cannabis infused products includes a cannabis infused product; a label format having a barcode providing a unique identifier; and a label indicia representing cannabis-related specifics of the cannabis infused product, wherein the label indicia is associated with the unique identifier; and a database providing regulatory tracking information associated with the unique identifier.

In another aspect of the present invention, a labeling format for preventing accidental and/or undesirable ingestion of a cannabis infused product includes an edible label sheet disposed on the cannabis infused product, wherein the edible label sheet has a warning logo adapted to connote to a consumer that an associated food product has at least a portion of cannabis infused therein.

In yet another aspect of the present invention, a labeling system for preventing accidental and/or undesirable ingestion of a plurality of cannabis infused products, each cannabis infused product having the above-mentioned label format further includes a container retaining the plurality of cannabis infused products; a removable packaging housing the plurality of cannabis infused products in the container; and a label format image adhered to the removable packaging, where the label format image comprises the, warning logo, the barcode, the plurality of label indicia, and the color coding of the label format.

These and other features, aspects and advantages of the present invention will become better understood with reference to the following drawings, description and claims.

### BRIEF DESCRIPTION OF THE DRAWINGS

FIG. 1 is a top plan view of an exemplary embodiment of the present invention, shown in use;

FIG. 1A is a schematic illustration of an exemplary embodiment of the present invention, demonstrating indicia of potency levels;

FIG. 2 is a top plan view of an exemplary embodiment of the present invention;

FIG. 3 is an exploded plan view of an exemplary embodiment of the present invention;

FIG. 4 is a perspective view of an exemplary embodiment of the present invention, shown in use; and

FIG. 5 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a label format for ingestible products and method for preventing accidental and/or over-ingestion of cannabis infused products, thereby identifying the THC and CBD levels of cannabis infused products, providing a clear edible warning label that can be placed directly on the infused edible itself; providing visible identification of cannabis infused food products; and providing descriptive content of cannabis-related information.

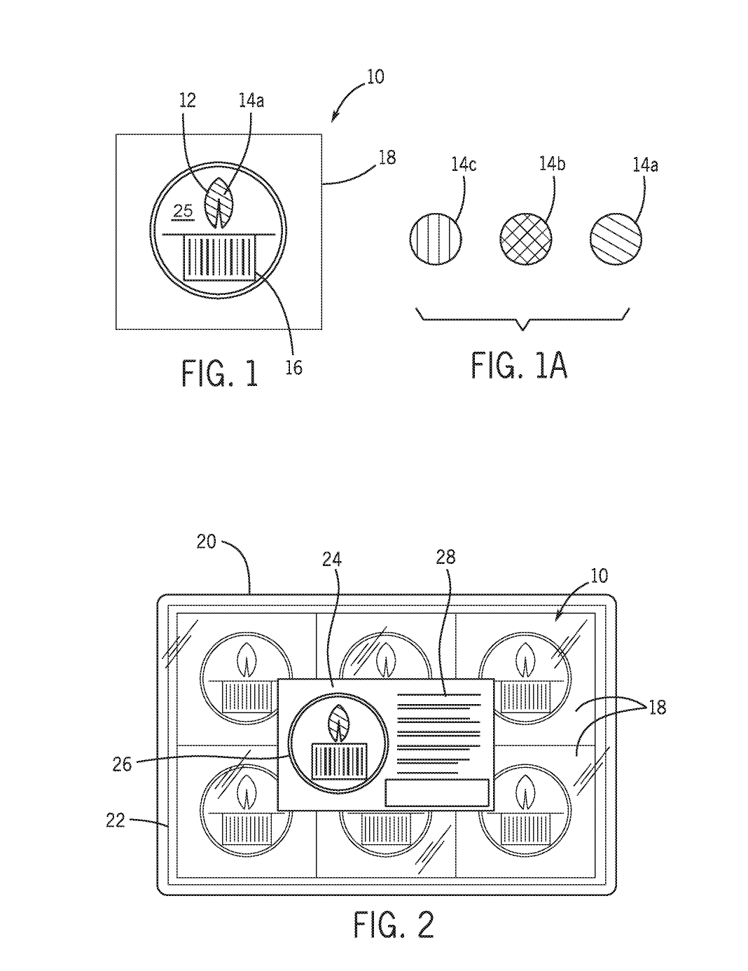

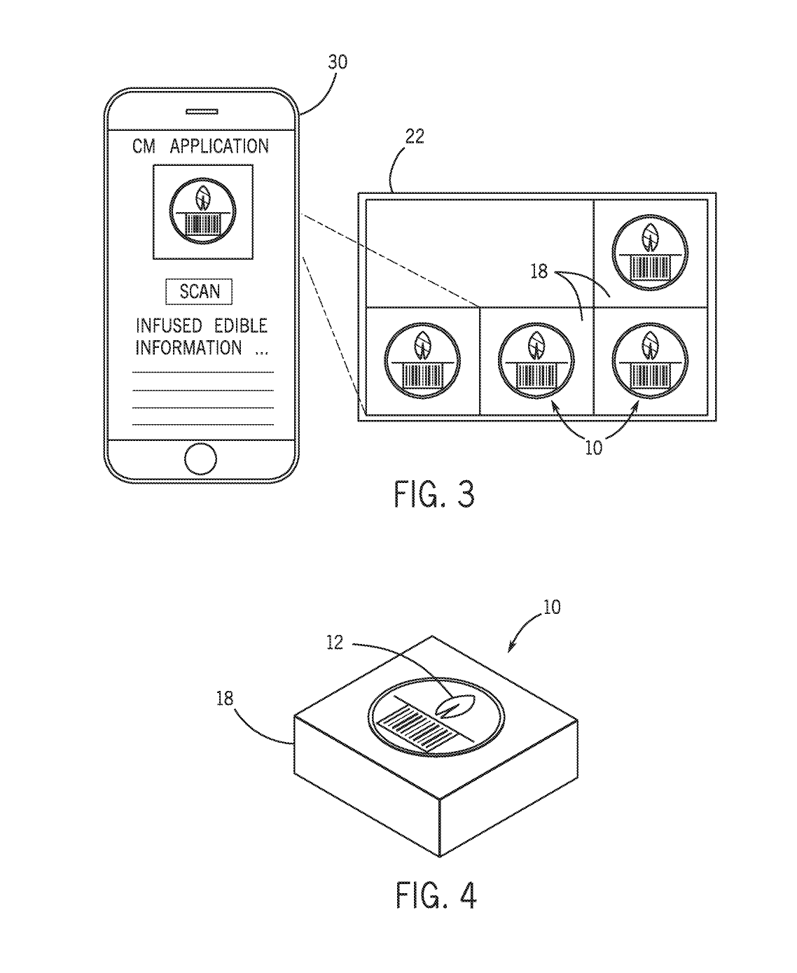

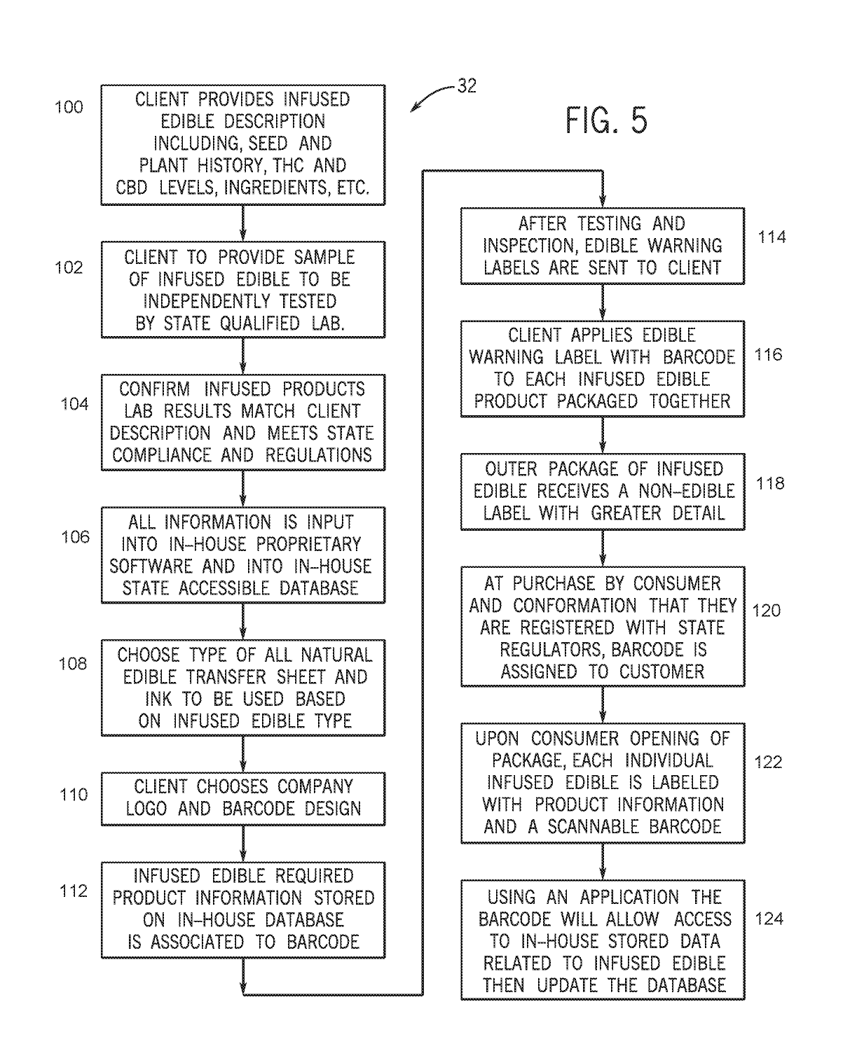

Referring to FIGS. 1 through 5, the present invention may include labeling system embodying a label format 10 and a method for preventing accidental and/or over-ingestion of cannabis infused products 18. The label format 10 may include a label sheet 25 made from edible material that provides surface area adapted to receive printable information, such as a logo 12, color coding 14, UPC coding 16, and, in certain embodiments, label indicia 28. At least one side of the label sheet 25 may provide an edible adhesive element for adhering to cannabis infused products 18. The color coding 14 may include a first color 14a designated for a first level of potency, such as green for a lower potency, a second color 14b designated for a second level of potency, such as orange for a medium potency, and a third color 14c for a third level of potency, such as red for a higher level of potency.

Typically, the cannabis infused products 18 are provided in containers 22 having removable packaging 20. The removable packaging 20 may provide a label format image 26. Each underlying cannabis-infused product 18 may have disposed, or otherwise provided, the label format 10 directly thereon. In certain embodiments, the label format image 26 and an associated label sheet 24 and printable information is non-edible and may contain more information than the underlying label format 10 of the individual cannabis-infused products 18, such as a more expansive label indicia 28.

The labeling system embodying the label format 10 and method thereof may include an initial vetting process using in-house software that is constantly updated with relevant state regulatory, manufacturing and other participating company information. When a client requests service, the system checks the status of the requesting client with the state: if the client is either not registered or in good standing the system will reject service; or if the requesting client is registered and in good standing, the system will allow post-vetting steps.

Once confirmed the requested information regarding seed and plant history is then input into the system. If any information provided by the requesting client is not confirmed, the requesting client will receive automated email requesting corrections within a specific time period that, once confirmed, will continue to next steps, which includes acceptance of third party lab results.

Third party lab results received from requesting clients may be checked in-house. Requesting client may go through an automated identical process similar to the one stated above, wherein the system will continue to next steps after appropriate confirmation.

Each label format 10 may be ingestible, print ready and have predetermined chemical components. Each label format 10 may be tested for chemical alteration, wherein the system detects a change in either THD or CBS levels due to edible logo edible ink, or transparent edible ink, whereby the label will be rejected. In such case, the system may then request adjustments be made accordingly. Once such adjustments have been confirmed, the system may approve next steps, including transparent infrared barcode reader application.

Once the system approved a sample, the edible warning label 10 may be printed, so that any reader application tests that scan results can match input data for pushing the process forward, including client sign off.

The system may send the requesting client email for approval. Once approved, the system will update the state accessibly database, and once updated, the system will move to batch print process.

The warning logo 12 may be a simple circle with “cm” letters in the center using FDS approved edible inks. The warning logo 12 may be adapted to connote to a consumer that an associated food product contains, in part, cannabis or components thereof. The print process may be modified via proprietary edible ink cartridges, including use with transparent barcodes.

In certain embodiments, the system may include at least one computer 30 with a user interface. The computer 30 may include at least one processing unit and a form of memory including, but not limited to, a desktop, laptop, and smart device, such as, a tablet and smart phone. The computer 30 includes a program product including a machine-readable program code for causing, when executed, the computer to perform steps. The program product may include software which may either be loaded onto the computer 30 or accessed by the computer 30. The loaded software may include an application on a smart device. The software may be accessed by the computer 30 using a web browser. The computer 30 may access the software via the web browser using the internet, extranet, intranet, host server, internet cloud and the like.

The computer 30 takes advantage of built in infrared technology so that the program product is designed to remove the need to use blue light that is required for standard transparent printing. The program product may keep track of the amount of ingested infused product 18 and update the database making it available to state, lab, dispensary, patient, and the like. The program product may also be adapted warn a consumer if intake exceeds recommended use (even if used in recreational approved states).

The embodied method 32 may include that a client provides infused edible description and associated label indicia 28. The label indicia 28 may provide or represent a data set for the portion of cannabis infused in the cannabis infused product 18. The data set may include seed and plant history, THC and CBD levels, ingredients and the like, in step 100. Such label indicia 28 may determine and confirm infused edible size and makeup and/or type of edible based on client description. In step 102, the client may provide sample of infused eligible product 18 to be tested independently by a state-qualified lab, whereby the system requests lab test confirmation from client and official lab results directly from the associated lab facility. Then, in step 104, the system may compare client and lab result information so as to confirm infused product 18 lab results meet state compliance and regulations. Step 104 may include requesting origin of seed and plant documented history, confirmation of receipt of information from client with state records, reconfirmation of the THC and CBD levels via in-house testing, informing client of all findings, the client to sign off on all state required documents and other related paperwork. In step 106, all above-mentioned information may be input into in-house proprietary database having state or regulatory required tracking information and infused product content loaded into in-house state accessible database. In step 108, choose type of all natural FDA approved, or all natural FDA approved organic or non-organic edible transfer sheet 24, or laser depending on food product type. In step 110, client may choose print ready company logo 12 and barcode 16 holding all required information and label indicia 28, which in turn is stored on in-house proprietary database, in step 112. The proprietary database may include food grade print software adapted to prompt design for food grade on in-house designed food grade edible printer and to prompt design for proprietary food grade transparent or opaque barcode print cartridge. To wit, edible warning labels 10 complete with state required information, THC, CBD seed to plant origin, lab results, and tracking information may be printed onto transparent or opaque edible barcode 16 within or near a company logo 12. In step 114, the printed product and paper may then tested for any chemical alteration of edible warning label in preparation of placement directly on food product 18. Warning logo 12 and transparent or opaque barcode 16 may then be tested for reading results for an end recipient computer 30 that allows consumer to use infrared base cell phones to scan and read the edible warning label 10 supplied barcode 16. Quality control check may be done in temperature controlled, food safe environments. Then the edible warning labels 10 may then packed to be sent to client.

In step 116, the client applies the edible label 10 with barcode 16 to each infused edible product 18 separately or packaged together in a container 22. If packaged together, the outer container 22 receives a non-edible label 26 with greater detail, including label indicia 28, in step 118. In step 120, the consumer, at purchase and upon confirmation that they are registered with state regulators, may be assigned to an associated barcode 16. In step 122, upon consumer opening the container 22 and/or its removable packaging 20, each individual infused edible product 18 is labeled with product information and a readable barcode 16. In step 124, the computer 30 may allow access to in-house database related to each associated infused product 18 then updated in the database.

Alternatively, the present invention can be manufactured in a non-edible form to serve as a external warning label for liquid based infused products.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A labeling format for preventing accidental and/or undesirable ingestion of a cannabis infused product, comprising an edible label sheet disposed on the cannabis infused product, wherein the edible label sheet comprises a warning logo adapted to connote to a consumer that an associated food product has at least a portion of cannabis infused therein.

2. The labeling format of claim 1, wherein the warning logo further comprises a color coding having one of a plurality of colors, each color associated with a level of potency the portion of cannabis.

3. The labeling format of claim 2, wherein the label sheet further comprises a barcode providing a unique identifier.

4. The labeling format of claim 3, wherein the label sheet further comprises a plurality of label indicia representing a data set for the portion of cannabis, wherein the plurality of label indicia is associated with the unique identifier.

5. The labeling format of claim 4, wherein the data set includes seed and plant history and THC and CBD levels of the portion of cannabis.

6. The labeling format of claim 5, wherein the barcode provides regulatory tracking information associated with the unique identifier.

7. A labeling system for preventing accidental and/or undesirable ingestion of a plurality of cannabis infused products, each cannabis infused product having the label format of claim 6, further comprising:
a container retaining the plurality of cannabis infused products;
a removable packaging housing the plurality of cannabis infused products in the container; and
a label format image adhered to the removable packaging, where the label format image comprises the, warning logo, the barcode, the plurality of label indicia, and the color coding of the label format of claim 6.

8. A labeling system for preventing accidental and/or undesirable ingestion of cannabis infused products, comprising:
a cannabis infused product;
a label format, comprising:
a barcode providing a unique identifier; and
a label indicia representing cannabis-related specifics of the cannabis infused product, wherein the label indicia is associated with the unique identifier; and
a database providing regulatory tracking information associated with the unique identifier.
